(19) United States
(12) Patent Application Publication
(10) Pub. No.: US 2024/0017088 A1
(43) Pub. Date: Jan. 18, 2024

(54) LOAD-REGULATED IMPLANTABLE OPTICAL MICRO DEVICE

(71) Applicant: AARHUS UNIVERSITET, Aarhus C (DK)

(72) Inventors: Amin Rashidi, ZG Delft (NL); Farshad Moradi, Risskov (DK); Milad Zamani, Risskov (DK)

(21) Appl. No.: 18/023,574

(22) PCT Filed: Sep. 1, 2021
(86) PCT No.: PCT/EP2021/074096

§ 371 Date: Feb. 27, 2023

(30) Foreign Application Priority Data
Sep. 1, 2020 (EP) 20193848.7

(51) Int. Cl. A61N 5/06; H02J 50/15

(52) U.S. Cl. CPC A61N 5/0622; H02J 50/15; A61N 5/062; A61N 5/0601; H02J 2310/23; A61N 2005/0626; A61N 2005/0652; A61N 2005/0663

(57) ABSTRACT

A micro device, such as a brain dust, is arranged for implantation into biological tissue. A power management unit receives a wireless signal, e.g. an ultrasonic signal, from an external source and generates an electric power output accordingly. The micro device has a plurality of electrical power consuming components powered by the electric power output, especially comprising one or more controllable light sources, e.g. micro LEDs which can be controlled to generate light in a time-varying manner, such as for optogenetics or for optical release of a drug. To increase power efficiency, an electric regulator circuit regulates electric current applied to the controllable light source to provide a predetermined total electric load of the power management unit. This provides an optimal efficiency of transferring power to the plurality of electrical power consuming components with a minimal requirement of volume of the circuit which provides impedance matching for optimal efficiency.

### CROSS-REFERENCE TO RELATED APPLICATIONS

This application is the U.S. National Stage of PCT/EP2021/074096 filed on Sep. 1, 2021, which claims priority to European Patent Application 20193848.7 filed on Sep. 1, 2020, the entire content of both are incorporated herein by reference in their entirety.

### FIELD OF THE INVENTION

The present invention relates to the field of implantable micro device. More specifically, the invention provides a micro device for implantation into biological tissue, e.g. the brain, such as a so-called brain dust. Specifically, the invention provides a micro device with a power efficient electric circuit for controlling at least one light source for optogenetics, light-driven drug delivery, or photodynamic therapy.

### BACKGROUND OF THE INVENTION

Micro devices suitable for implanting into biological tissue are typically electrically powered with one or more sensors and/or one or more actuators, and typically such micro devices provide data communication with an external device. However, all of these functions require electric power, which is challenging for very compact dimensions of the micro device, e.g. the so-called deeply seated dusts for implantation into brain tissue. A battery in such micro devices, if present at all, has a very limited capacity, and any wireless power transfer still has a limited power capacity due to safety concerns. Thus, there is a need for extremely power-efficient solutions for all functions in the micro device. The electrical loads in such devices can vary from a few μW to several mW depending on their functionality.

Especially, micro devices comprising a light source for e.g. optogenetics, drug delivery triggering, or photodynamic therapy are vulnerable with respect to electric power, since to provide light for the desired functionality, a considerable amount of electric power is required at the time when the light source is active. For wireless power transfer by means of ultrasonic signals from an external device, an impedance matching circuit can be used to provide an optimal power transfer if the impedance of the power demanding circuit is known. However, in case of a highly variable loads over time, a poor overall power transfer efficiency is achieved, if a fixed impedance matching circuit is used.

US 2017/0117753 A1 describes one possible solution to this problem, namely providing a reconfigurable matching circuit. However, this introduces disadvantages, namely a significant increase in size of the micro device, since such reconfigurable matching circuit requires use of a plurality of rather large capacitors to allow switching between various matching impedances. Further, the switching between matching impedances may require 2-way communication with an external device, thus in itself requiring the 2-way communication functionality in the micro device and this communication itself requires more power and adds complexity to the system.

Thus, for very compact micro devices, such as brain dusts, there is a need for a compact and at the same time power efficient solution to provide the highest possible electrical power out of the ultrasonic waves at the micro device.

### SUMMARY OF THE INVENTION

Following the above, it may be seen as an object of the present invention to provide a compact and yet highly power efficient micro device.

In a first aspect, the invention provides a micro device, such as a so-called brain dust, arranged for implantation into biological tissue, the micro device comprising
a power management unit arranged to receive a wireless signal, such as an ultrasonic signal, from an external source and to generate an electric power output accordingly,
a plurality of electrical power consuming components powered by the electric power output, the power consuming components comprising at least one controllable light source, such as a micro LED, arranged to generate light in a controllable time-varying manner, such as for optogenetics, light-driven drug delivery, photo-dynamic therapy etc., and
an electric regulator circuit arranged to regulate electric current applied to one controllable light source so as to provide a predetermined total electric load of the power management unit for optimal efficiency of transferring power to the plurality of electrical power consuming components.

Such micro device is advantageous, since it provides a high power transfer efficiency of the received wireless electric power even though only a limited number of components with a small size can be used. This is obtained by the load regulation approach which eliminates the need for a bulky and complicated adjustable impedance matching circuit which suffers from multiple large capacitors to implement the adjustable impedance for matching the electric load. This is important in an implanted micro device with multiple functionalities on board, e.g. light sources, drug delivery, electric stimulation electrodes as well as various sensing properties, data processing and data transmission, which in general causes the electric power consumption to vary significantly over time. However, this is taken into account ensuring a constant total electric load, thereby allowing optimal efficiency using a single impedance matching circuit. Thus, at the same time, the micro device can be designed with compact dimensions and with a high electric power efficiency. In this way the micro device is highly suitable as a so-called brain dust for implantation into brain tissue to allow e.g. optical stimulation, neural activity sensing, as well as other functions wirelessly controlled from an external device. In essence, the load regulating circuit operates by regulating current through the at least one light source, e.g. LED, so as to ensure a constant total current drawn from the power management unit.

Furthermore, the invention offers more advantages at no- or low-extra cost including the longevity management of the energy stored at the micro device, e.g. demodulation of time-encoded downlink data, dual-control of the loads using one set of data, and overvoltage protection.

In the following, preferred features and embodiments of the first aspect will be described.

In preferred embodiments, the electric regulator circuit is arranged to regulate electric current applied to the at least one controllable light source in a closed-loop manner, so as to regulate a total current applied to all of the electrically power consuming components. In this way, the load regulator circuit adapts to changes in power consumption by the single power consuming components and serves to provide a constant total current delivery by the power management unit. Preferably, the micro device has an impedance matching circuit connected to the power management unit to provide optimal power efficiency, wherein the impedance matching circuit has fixed components. This provides maximum efficiency with a minimal space required, since only one single matching circuit is required compared to prior art adjustable matching circuits.

Preferably, the at least one controllable light source comprises a micro Light Emitting Diode (LED) connected to a driver circuit. Especially, the driver circuit may switch on or off the LED in a controllable manner according to an input, or the driver circuit may be arranged to vary intensity of the LED in a controllable manner by controlling current through the LED according to an input.

Preferably, the plurality of electrically power consuming components comprises a wireless receiver arranged to control the at least one controllable light source, especially such as for controlling light intensity of the at least one controllable light source. The wireless receiver may be an RF receiver or ultrasonic receiver.

Preferably, the micro device comprises a wireless receiver, e.g. an ultrasonic receiver, separate from the power management unit, and being arranged to receive data for controlling at least one of the plurality of electrically power consuming components, such as the at least one controllable light source.

In some embodiments, the electrical regulator circuit is arranged to demodulate time-encoded downlink data embedded in the wireless signal received from the external source, and wherein the demodulated downlink data is used to control power consumption of electrically power consuming components.

The electrical regulator circuit is preferably arranged to disable at least one power consuming component when there is lack of power for energy longevity management at the micro device.

The electrical regulator circuit may be arranged to demodulate a time-encoded downlink data embedded in the wireless signal received from the external source, and wherein the demodulated downlink data is used to control power consumption of electrically power consuming components.

Especially, the at least one controllable light source may be arranged for optogenetic stimulation and/or photodynamic therapy, and/or optic triggering of release of a drug from a drug container.

The plurality of electrical power consuming components may comprise a second controllable light source, wherein the first and second controllable light sources are arranged to generate light at different wavelengths, such as the first and second controllable light sources being arranged for optogenetics or photodynamic therapy at different wavelengths of light. Especially, the first controllable light source is arranged for one of: optogenetics, photodynamic therapy or for optical triggering of drug delivery, and wherein the second controllable light source is arranged for one of: optogenetics, photodynamic therapy or for optical triggering of drug delivery. Preferably, the electric regulator circuit is arranged to regulate electric current applied to both of the first and second controllable electric light sources so as to provide the predetermined total electric load of the power management unit for optimal power efficiency. The electric regulator circuit may comprises a load regulator arranged to drive the first controllable light source, and a current Digital to Analog Converter arranged to drive the second controllable light source. Especially, the load regulator may be arranged to adapt a current to the first controllable light source in order to provide said predetermined total electric load of the power management unit. Especially, the load regulator circuit may be arranged to drive the first controllable light source, when electric power is available from the power management unit. Especially, the load regulator circuit may be arranged to drive the second controllable light source based on a received command.

In some embodiments, the micro device comprises a third controllable light source arranged for optogenetics, photodynamic therapy or for optical triggering of drug delivery. Especially, the first and second controllable light sources are arranged for optogenetics or photodynamic therapy at different wavelengths of light, and wherein the third controllable light source is arranged for optical triggering of drug delivery.

In some embodiments, the micro device comprises an electrode arranged for controllable electric stimulation of biological tissue, and wherein the at least one controllable light source is arranged for optogenetics, such as for providing hybrid stimulation of biological tissue using both optogenetics and electric stimulation, optically enhanced electrical stimulation. The biological tissue may in this case be any of brain tissue, any location in the central nervous system, or peripheral nerves or muscular tissue. E.g. such hybrid stimulation may be applied for treatment of chronic pain.

In preferred embodiments, the power management unit is arranged to receive an ultrasonic data from an external source and to generate both the electric power output and the downlink data, accordingly, e.g. by means of a piezoelectric receiver arranged to receive an ultrasonic signal transmitted through the biological tissue. However, alternatively or additionally, the power management unit may comprise a radio frequency (RF) antenna arranged to receive an RF electromagnetic signal transmitted through the biological tissue.

In some embodiments, the plurality of electrically power consuming components comprises at least one sensor arranged to measure neural activity or a physical parameter of the biological tissue, such as temperature, pressure or the like. Especially, the sensor may comprise a Local Field Potential sensor or a single neural cell sensor. Especially, the micro device may comprise a wireless transmitter arranged to transmit data in a wireless format to an external receiver, such as by means of ultrasonic backscattering, such as data indicative of the neural activity or the physical parameter measured by a sensor. Further, the micro device may comprise a first sensor arranged to sense neural activity, and a second sensor arranged to sense a physical parameter of the biological tissue, such as temperature, pressure or the like.

In preferred embodiments, the power management unit, and the electric regulator circuit are implemented on an integrated circuit die. Especially, being configured in conjunction with a piezoelectric receiver and first and second light sources.

Especially, the micro device for implantation into biological tissue, such as brain tissue, muscular tissue or the like, such as the micro device having outer dimensions occupying a total volume of less than 5 mm³, such as less than 2 mm³ or even less than 1 mm³.

Especially, the ultrasonic transmitter may be arranged to generate an ultrasonic signal with a frequency in the range of at least 100 kHz to a few MHz, or higher.

The light sources may be micro LEDs with a suitable wavelength of light, such as known by the skilled person.

The micro device preferably comprises a processor capable of providing at least a minimum of data processing. Especially, the processor may be capable of executing a neural network algorithm for deciding about when and how to apply optical and/or electrical stimulation. Further, or alternatively, the processor may be capable of processing sensed data so as to reduce the amount of data to be transmitted from the micro device, e.g. to derive event based data, e.g. event based neural data, for transmission from the micro device.

In preferred embodiments, the power management unit comprises an ultrasonic receiver transmitter system arranged to receive power in an ultrasonic signal, and wherein the embedded ultrasonic transducer, e.g. piezoelectric transducer, is arranged to transmit an ultrasonic signal with data from the micro device represented therein. E.g. as a back scattered ultrasonic signal to an external ultrasonic detector system. Such embodiments utilizes a combination of ultrasound power transmission to the embedded electronics, and at the same time allow use of back scattered ultrasonic signals as communication to the external signal receiver system.

In some embodiments, a drug delivery system is embedded in the dust, e.g. for controllable delivery of a drug in response to a signal received from an external optical or ultrasonic transmitter.

In some embodiments, the electric regulator circuit is configured for generating controllable voltages at the output of the power management unit. Especially, the controllable voltages at the output of the power management unit may be used for controlled electrical stimulation, such as enhancing stimulation efficiency by concurrent optical and electrical stimulation.

In some embodiments, the micro device comprises an overvoltage protection circuit, such as an overvoltage protection circuit integrated with the electric voltage regulator or other circuit in the micro device.

In some embodiments, the micro device comprises a plurality of light sources, and wherein electric load regulator comprises respective current weights to determine a weighting of current to be applied to the individual light sources.

The power management unit preferably comprises a piezoelectric transducer arranged to receive an ultrasonic signal and to generate the electric power output accordingly.

The micro device may have a total volume of less than 1 mm³, such as less than 0.5 mm³, such as less than 0.2 mm³. A micro device has been tested with a piezoelectric ultrasound power receiver and with a blue and a red LED, and with dimensions 500×500×500 μm, i.e. with a volume of 0.125 mm³.

In an advantageous embodiment of the invention, the size of the micro device is small, and for implantation purposes, it may be preferred that the micro device is as small as possible. In preferred embodiment, the dimensions of the micro device is within 1×1×1 mm (height×length×width), such as within 500×500×500 μm, such as within 400×400×400 μm, such as within 300×300×300 μm, such as within 200×200×200 μm and in some embodiments it may be seen as most preferably to be within 100×100×100 μm. It is to be understood that the micro device may preferably be even smaller than 100×100×100 μm in case the actual manufacturing technologies chosen allows to.

In preferred embodiments, the micro device has a total volume of less than 2 mm³, preferably less than 1 mm³, preferably less than 0.7 mm 3, such as less than 0.5 mm³.

In some embodiments, the micro device have non-uniform height, length and width. Especially, the height, length, and width dimensions may be such as 200×150×100 μm , or such as 150×150∴100 μm, or such as the micro device having a height within 0.5-1.5 mm, a length of 0.5-1.0 mm, and a width of 0.3-0.7 mm.

In a second aspect, the invention provides a method for managing power consumption in a micro device arranged for implantation into biological tissue, the method comprising
providing a wireless signal from an external source to a power management unit of the micro device,
powering electrically power consuming components of the micro device based on electric power from the power management unit, and
regulating an electric current applied to at least one controllable light source by means of an electric regulator circuit so as to provide a predetermined total electric load of the power management unit for optimal power efficiency.

The invention may be exploited by the following design methodology steps:
1) Defining the total required power at the micro device including the load of at least one light source for optogenetics, photodynamic therapy or light driven drug delivery.
2) Designing a matching circuit for the targeted optimal load, i.e. optimal load's voltage and current, e.g. matching circuit for matching of an ultrasonic transducer power receiving transducer to provide optimal power efficiency for the targeted optimal load, and
3) Designing the electric load regulator circuit for optimal load by driving the most power demanding light source (in the case that there are several light sources).

Following these design steps, it has been found that an increased power efficiency can be obtained without the need for a bulky variable matching circuit.

In a third aspect, the invention provides a system, such as a computer to brain interface, such as a medical therapy or treatment system, comprising
a plurality of micro devices according to the first aspect, such as in the form of a so-called neural dust, and
an interface system comprising
a wireless signal transmitter, such as an ultrasonic signal transmitter, for transmitting a wireless signal to the micro devices for powering the micro devices and controlling the electric power consuming components of the micro devices, and
a wireless control signal transmitter, such as an ultrasonic control signal transmitter or an electromagnetic radio frequency (RF) transmitter, for transmitting a wireless control signal for individually controlling the at least one controllable light source(s) of each of the individual micro devices.

Especially, the whole of or part of the interface system may be arranged for implantation into biological tissue, e.g. brain tissue. Specifically, the interface system may comprise a first part arranged for position external to biological tissue and a second part being arranged for position in biological tissue in proximity of the plurality of micro devices, wherein the first part comprises a computer for interface to an external computer programmed for controlling the function of the micro devices via the first and second parts of the interface system.

Specifically, the interface may further comprise a wireless receiver arranged to receive a wireless data signal, such as an electromagnetic RF signal or an ultrasonic signal, e.g. an ultrasonic backscattered signal, from one or more of the micro devices, wherein the one or more micro devices comprises a sensor arranged to sense a physical parameter and to transmit wireless data signal accordingly. This sensor may be one or more of: a temperature sensor, a neural activity sensor such as a Local Field Potential sensor, a single neural cell sensor, or a pressure sensor.

In a fourth aspect, the invention relates to the use of the micro device or system according to the first or third aspects. Especially, use of the micro device or system for treatment or therapy on a living person or animal. Especially, the micro device is a so-called neural dust arranged for implantation into brain tissue and being arranged for treatment or therapy of one or more diseases and/or pain. The micro device may be capable of single or double wavelength optical therapy or optogenetics for neuromodulation, and this may be combined with electric stimulation of the brain tissue to provide electric neuromodulation. In embodiments where the micro devices comprise an electric neural sensor, a closed loop control of the applied treatment or therapy may be provided.

In one embodiment, the micro device or system according to the first or third aspects is used for treatment of pain, e.g. chronic pain, by implanting one or more micro device into or near peripheral nerves or input muscular tissue. The micro device for this use may be capable of single or double wavelength optical therapy or optogenetics for neuromodulation, and this may be combined with electric stimulation of the nerve or muscular tissue to provide electric neuromodulation. In embodiments where the micro devices comprise an electric neural sensor, a closed loop control of the applied treatment or therapy may be provided.

This aspect of the invention may be particularly advantageous for treating illnesses or pathologies such as, but not limited to chronic pain, depression, movement disorders, Parkinson's disease, Alzheimer's disease, epilepsy, blindness. The invention, as it revolves around measuring and providing signals and stimulus to and from the brain, may be suitable for treating a plurality of ailments relating to chemical, hormonal or electrical imbalances and may furthermore be used to transmit sensory or motor signals from the peripheral nervous system (somatic and autonomous system), which are not sufficiently transferred to the central nervous system, either due to trauma, prenatal diseases or other diseases related to the nervous system.

The same advantageous mentioned for the first aspect apply for the second, third and fourth aspects as well. The individual aspects of the present invention may each be combined with any of the other aspects. These and other aspects of the invention will be apparent from the following description with reference to the described embodiments.

### BRIEF DESCRIPTION OF THE FIGURES

The invention will now be described in more detail with regard to the accompanying figures. The figures show one way of implementing the present invention and is not to be construed as being limiting to other possible embodiments falling within the scope of the attached claim set.

FIG. 1a and FIG. 1b illustrate a micro device embodiment and a system embodiment;

FIG. 2a illustrates a power management unit for receipt of power from an ultrasonic signal, while FIG. 2b illustrates the corresponding Thevenin equivalent diagram;

FIG. 3 illustrates a graph related to FIGS. 2a and 2b;

FIG. 4 illustrates a block diagram of an embodiment with one LED;

FIG. 5 illustrates a block diagram of an embodiment with two LEDs;

FIG. 6 illustrates one example of a circuit for the embodiment of FIG. 4;

FIG. 7 illustrates a block diagram of an embodiment with sensor and stimulation electrodes;

FIG. 8 illustrates one example of a circuit for the embodiment of FIG. 7;

FIG. 9 illustrates steps of a method embodiment;

FIG. 10 illustrates a block diagram of a load regulator with current weights for multi LEDs;

FIG. 11 shows a schematic diagram of an experimental setup of a brain-computer interface system according to an embodiment of the invention;

FIG. 12, shows three graphs, representing transient measurement results from the experimental set up as shown in FIG. 11; and

FIG. 13 shows three graphs, representing measured acoustic intensity at the piezoelectric receiver, from the experimental set up as shown in FIG. 11.

### DETAILED DESCRIPTION OF AN EMBODIMENT

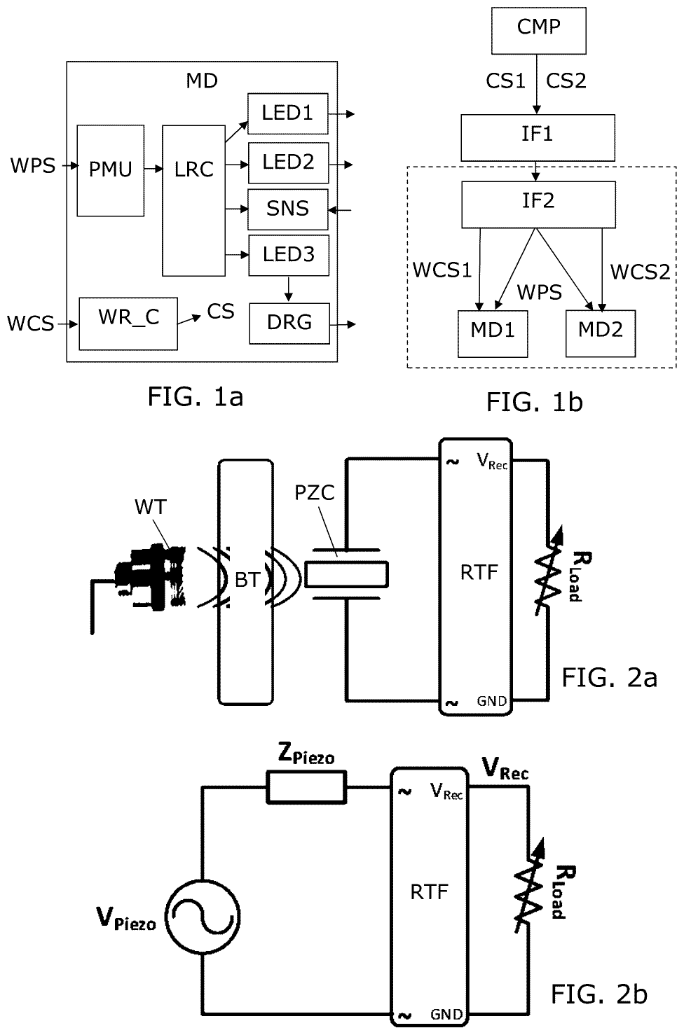

FIG. 1a illustrates a micro device MD, e.g. a so-called dust, embodiment which receives a wireless power signal WPS, preferably an ultrasonic signal, and a wireless control signal WCS, may be an ultrasonic and/or an electromagnetic RF signal. A power management unit PMU receives the wireless power signal WPS and generates a power output accordingly for powering all power consuming components of the dust, here including three light sources LED1, LED2, LED3, and a sensor SNS. A wireless receiver WR_C receives the wireless control signal WCS and provides a control signal or control signals CS accordingly, for control of the light sources LED1, LED2, LED3. Preferably, two light sources LED1, LED2 can generate optogenetics and/or optical therapy at different light wavelengths, while one light source LED3 can generate light for optically triggering drug delivery to surrounding biological tissue by providing light on a drug container DRG inside the dust (or positioned outside the dust).

An electric load regulator circuit LRC regulates currents to at least one of, preferably all of the light sources LED1, LED2, LED3, so that a predetermined total current is drawn from the power output of the power management unit PMU. In this way, a high power efficiency can be obtained in spite the power management unit PMU has a matching circuit with fixed electric components. The matching circuit is designed for optimal matching at the predetermined total current, and thus instead of varying the matching circuit to match the actual load, in this approach the load is adjusted to match the load for which the matching circuit is designed to provide optimal matching, and thereby achieving a high efficiency. Thus, with the proposed approach, a high electric efficiency can be combined with small space required for the necessary components.

FIG. 1b illustrates a system embodiment with a three layer approached for communication and powering of two implantable brain dusts MD1, MD2. A computer CMP outside a person's body communicates control signals CS1, CS2 for controlling function of the respective brain dusts MD1, MD2. The computer is connected to a first interface part IF1 to be placed on the head of a person, i.e. outside the skull. This first interface part IF1 communicates wirelessly with a second interface part IF2 which is arranged for implantation inside the skull of the person. This second interface part IF2 serves to provide power to the brain dusts MD1, MD2 by transmitting ultrasonic power signals WPS through the brain tissue to the implanted dusts MD1, MD2. Further, wireless control signals WCS1, WCS2 to the respective dusts MD1, MD2 are also transmitted, e.g. via ultrasonic signal or via electromagnetic RF signals. In this way, a computer CMP to brain interface can be implemented, and various functions of the dusts MD1, MD2, as the example device shown in FIG. 1a, can be individually controlled, e.g. to provide an electrical, drug and/or optical treatment, therapy, and/or to monitor neural activity.

FIG. 2a illustrates a wireless transmitter WT in the form of an ultrasound transmitter which transmits an ultrasonic signal through biologic tissue BT for powering a micro device implanted in the biologic tissue BT. The micro device has a piezoelectric receiver which receives the incoming ultrasonic signal and via a power management unit comprising a rectifying circuit RTF, e.g. a bridge rectifier, the power management unit can deliver electric load for powering a variable load R_{Load}. The principle is known as ultrasonically powered harvesting.

FIG. 2b illustrate a model of the circuit of FIG. 2a, namely a frequency- and load-dependent Thevenin equivalent in its steady state. Typically, for an implantable dust, the power carrier frequency is usually chosen in a frequency band known as inductive-band, so the imaginary part can be canceled using an on-chip capacitor.

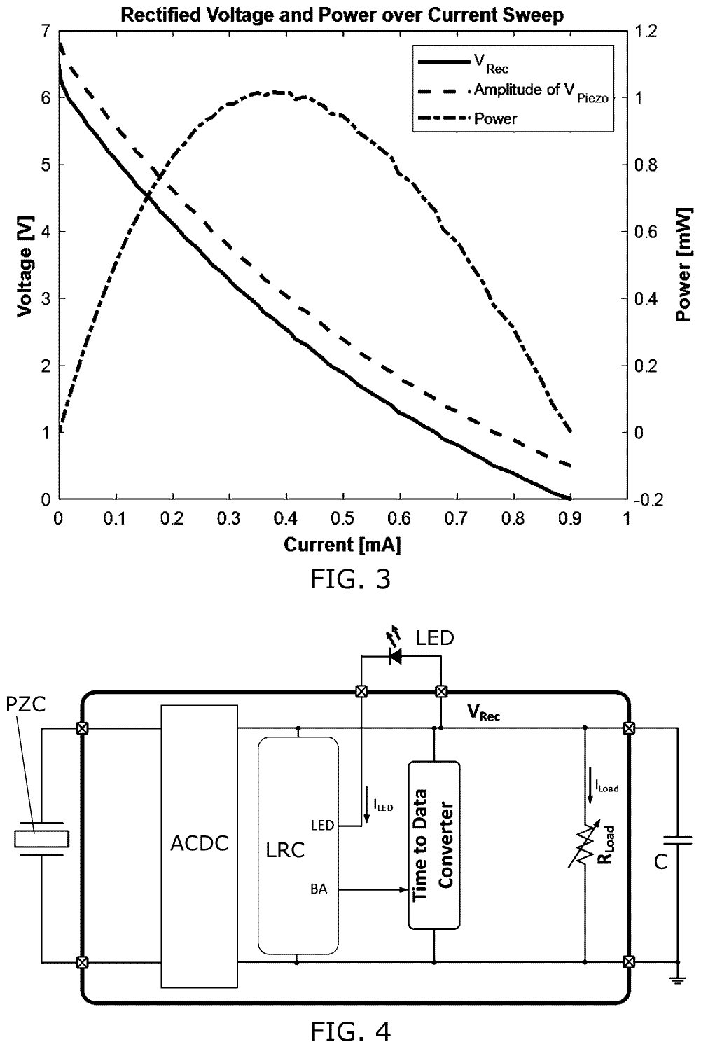

FIG. 3 illustrates a graph of measured rectified voltage V_{Rec} and the received power of a 500 μm×500 μm×500 μm piezo-crystal over a sweep of electrical current load. For this measurement, the crystal is exposed to ultrasonic waves with a power intensity of 7.2 mW/mm² at the crystal's resonance frequency, i.e. the frequency that the imaginary part of the equivalent impedance of piezoelectric crystal is zero, and an full-wave diode bridge rectifier is used for rectifying the signal at the piezoelectric crystal's terminals. A Keithley series source-meter is connected as the load in order to simultaneously sweep the current load and measure the voltage over it. The amplitude of the voltage V_{Piezo} of the piezoelectric crystal is higher than the rectified voltage by 2×VDF where VDF is the forward voltage of the diodes in the bridge rectifier. FIG. 3 shows how magnificent the electrical load can affect the amplitude of V_{Piezo} and the received power.

By changing the frequency of the power carrier in the inductive band and adding a capacitor for canceling the imagery part of the piezo impedance at the chosen frequency, the maximum received power can be designed for another electrical load. However, adaptive configuration of aforementioned parameters for a system with variable load, needs a bulky capacitor bank and a data communication link between the dust and the transmitter for adapting the frequency which comes at the price of extra complexity and silicon area.

FIG. 4 shows a block diagram of an example system architecture featured by the proposed load regulator LRC which overcomes the mentioned problems. In this architecture, the piezoelectric receiver transducer PZC is followed by an AC-DC converter ACDC which supplies the DC power, at voltage level of V_{REC} for driving a μLED and other variable loads, with equivalent resistance of R_{Load}and current of I_{Load}. A storage capacitor C is connected to the output of the rectifier. The proposed load regulator LRC regulates the deriving current of the μLED to result in an optimum total current load of Iₜₒₜₐₗ=I_{LED}+I_{Load}. The proposed load regulator LRC drives the LED just upon availability of ultrasonic power burst at the dust. It is favorable due to the fact that it helps extending the longevity of the energy stored at capacitor C for low-power-consumption continuous applications like neural recording. Furthermore, the load regulator LRC can generate a Burst Availability (BA) signal that can be used for deactivation of other power demanding none-critical circuits in absence of ultrasonic power burst. By encoding the downlink data in duration of some notches in the ultrasonic power burst, the BA signal can be used for detecting the notches. Thus, by feeding the BA signal to a time to data converter, the downlink data can be demodulated.

It is noteworthy, as illustrated in FIG. 3, that each current load results in a specific rectified voltage. Thus, by regulating the total current Iₜₒₜₐₗ to a specific value, the rectified voltage at the dust will be regulated too, and vice versa.

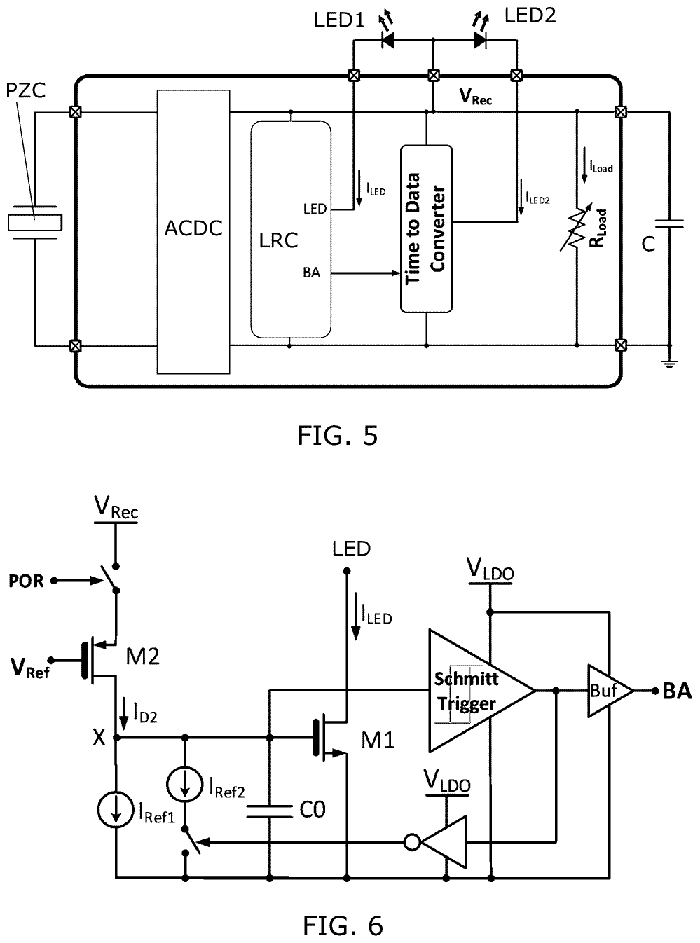

FIG. 5 illustrates another circuit embodiment with two lights source, namely two LEDs LED1, LED2, preferably generating light at different light wavelengths. One applications for such a system is dual optogenetics where two LEDs with two different wavelength are used for stimulation of two different opsins. Another application is a dust with two of the optogenetics, light driven drug delivery and photodynamic therapy. Here, the approach is dual control of the two LEDs LED1, LED2, and in this scheme the current for driving LED2 is derived by the current defined by the downlink data I_{LED2} and the current of LED1 also depends on the downlink data, i.e. I_{LED1}=Iₜₒₜₐₗ−I_{LED2}−I_{Load}). Considering that I_{Load} is negligible in comparison with optogenetic loads and Iₜₒₜₐₗ is the optimum total current for maximum power transfer defined by the ultrasonic link, it can be seen that with one set of data both I_{LED1} and I_{LED2} can be set in a power efficient way. Thus, based on the downlink data, the whole received energy can be used for driving one of the LEDs LED1, LED2 or can be divided between two LEDs.

FIG. 6 illustrates one possible circuit implementation of a load regulator LRC. V_{ref}, I_{Ref1}, and I_{Ref2} are reference voltage and currents. After Power On Reset POR, based on the level of the rectified voltage, the current through the transistor M2 I_{D2}, the voltage at node X, connected to the gate of transistor M1, and consequently the current through the transistor M1 I_{LED} is set. On the other hand, the level of I_{LED} defines the rectified voltage under a constant ultrasonic intensity at the dust, as illustrated in FIG. 3. Thus, a negative feedback loop is formed here to regulate the I_{LED} and V_{REC} to a specific value that can be mainly designed based on the values of V_{ref}, I_{Ref1}, and sizing of transistor M2. It is notable that for I_{D2} current levels smaller than the total current sank by the reference currents (i.e. I_{Ref1} and I_{Ref2}), the voltage at node X will be pulled down, and M1 turns off. Thus, in the absence of ultrasonic power burst at the dust, V_{REC} and consequently, I_{D2} and voltage at X drops and LED goes to zero. So, by feeding voltage at X to a Schmitt trigger, the availability of ultrasonic waves at the dusts can be monitored. Here, I_{Ref2} adds an extra hysteresis function to the circuit that tries to pull down the voltage on node X when M1 goes off in absence of ultrasonic waves in the dust. This eliminates avoids ringing in the voltage on node X and I_{LED}. The output of the Schmitt trigger is buffered to be used as BA signal by other circuits.

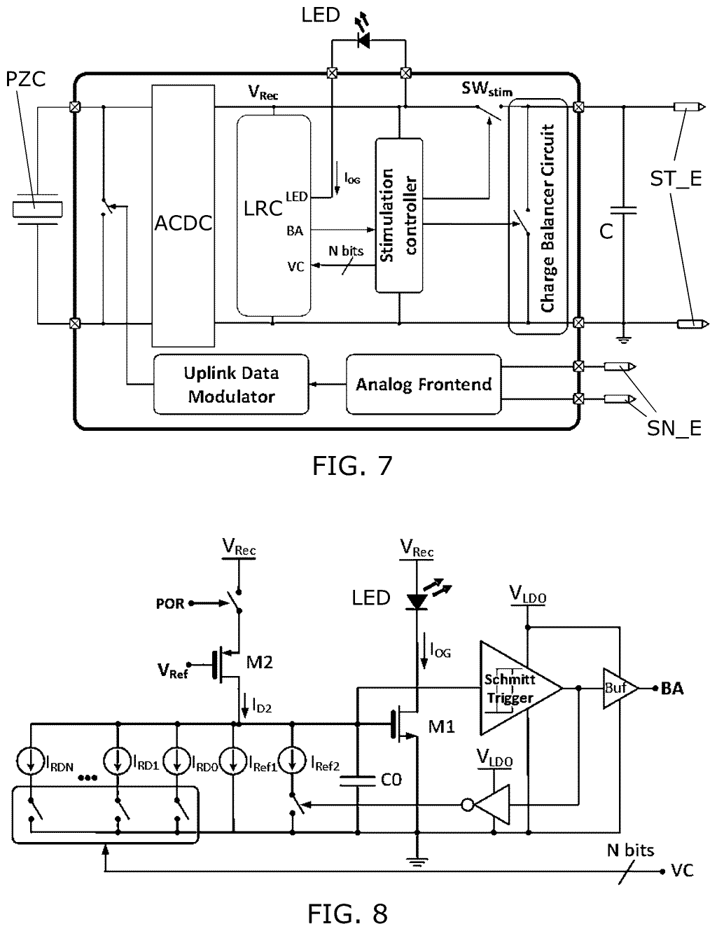

FIG. 7 illustrates a block diagram of an embodiment with one light source LED and a set of electric stimulation electrodes ST_E and a related charge circuit for stimulation of biologic tissue, e.g. neural stimulation. Further, this embodiment has a set of sensor electrodes SN_E, e.g. for neural activity sensing, and a related analog frontend circuit connected to an uplink data modulator that can modulate electric load of the piezoelectric crystal PZC to provide ultrasonic backscattering for transmission of data representing neural activity sensed by the sensor electrodes SN_E. In the architecture of FIG. 7, stimulation controller gets downlink data from the optogenetic regulator circuit LRC (through BA signal) and controls the stimulation amplitude and duration based on that data. The stimulation amplitude is set by changing the reference current of the optogenetic regulator. The duration of stimulation is controlled using a power switch SWₛₜᵢₘ. Furthermore, the stimulation controller should activate the charge balancer circuit after each stimulation. The charge balancer circuit can be either a power switch for passive charge balancing (simplest form) or an active charge balancer (higher safety).

FIG. 8 illustrates another example of a circuit implementation of the approach of FIG. 7 with some extra circuits for configuring the rectified voltage V_{Rec} for voltage-controlled electrical stimulation. The LED for optogenetics and the driver transistor M1 need a minimum overhead voltage, e.g. around 2.5 V for a red LED. So, I_{Ref1} is set to a value to ensure the minimum rectified voltage of 2.5 V. The reference currents I_{RD0}-I_{RDN} should be controlled based on Downlink data for higher rectified voltage, i.e. Stimulation Voltages.

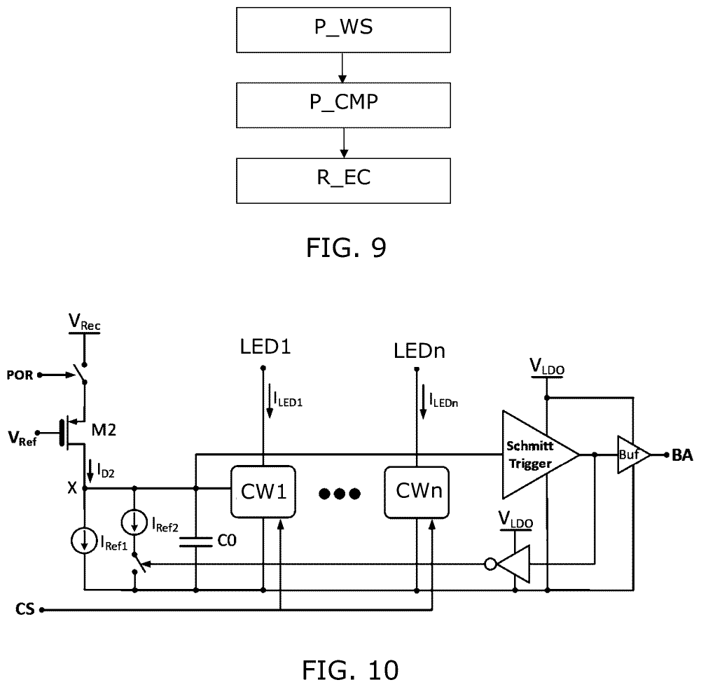

FIG. 9 illustrates steps of a method embodiment, i.e. a method for managing power consumption in a micro device, such as a brain dust, arranged for implantation into biological tissue, e.g. to be performed after the micro device has been implanted in biological tissue, e.g. brain tissue of a person. First step is providing P_WS a wireless signal from an external source to a power management circuit of the micro device, e.g. an ultrasonic signal. Next, powering electrically power consuming components of the micro device, e.g. one or more LEDs, based on electric power from the power management circuit. Finally, regulating an electric current applied to at least one controllable light source by means of an electric regulator circuit so as to provide a predetermined total electric load of the power management circuit for optimal power efficiency.

FIG. 10 illustrates one possible circuit implementation of a load regulator LRC for a multi-LED device. As in FIG. 6, V_{ref}, I_{Ref1}, and I_{Ref2} are reference voltage and currents. After Power On Reset POR, based on the level of the rectified voltage, the current through the transistor M2 I_{D2}, the voltage at node X, connected to all the respective current weight blocks CW1, CWn, of the LEDs LED1-LEDn, and consequently the current weight setting sets the current through each LED1-LEDn. A wireless receiver receives the wireless control signals CS accordingly, for controlling the weight of light sources LED1-LEDn. In the absence of ultrasonic power burst at the dust, V_{REC} and consequently, I_{D2} and voltage at X drops and I_{LED}s go to zero.

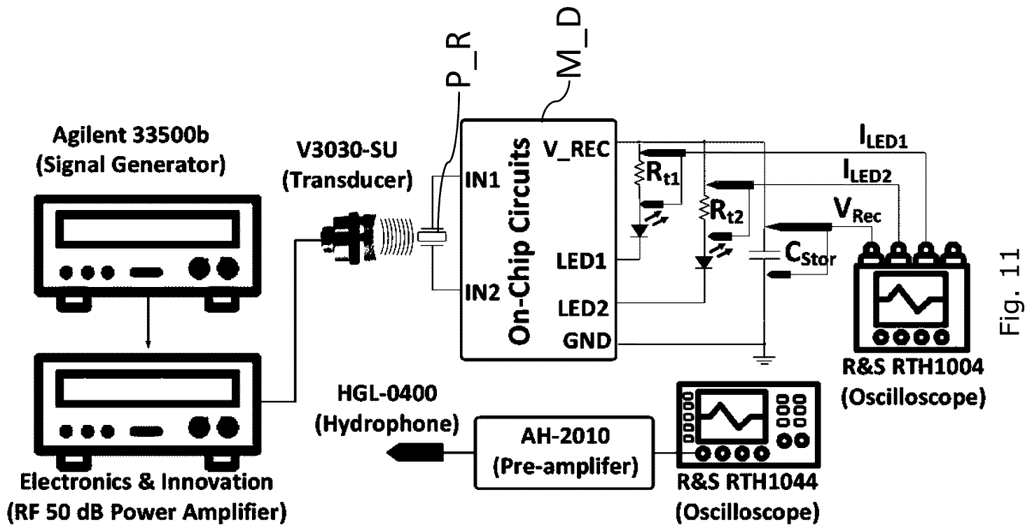

FIG. 11 shows a schematic diagram of an experimental setup of a brain-computer interface system, according to an embodiment of the invention. The inventors have set up a live experimental prototype, according to an embodiment of the invention, for the purpose of measuring dual-wavelength light, i.e. optogenetic signals, when the micro device M_D is powered by ultrasonic waves. The schematic diagram shows how the set up was built and how the experiment was performed. A 2.55 ms ultrasonic burst, including a series of duration-increasing notches is fed into an arbitrary signal generator, Agilent 33500b, and transmitted, as an ultrasonic power burst to the piezoelectric receiver P_R on the micro device M_D, through an amplifier, RF 50 dB power amplifier, and a transducer, V3030-SU. The transducer successfully powers two LED's LED1, LED2 and the light emitted from the LED's LED1, LED2 was measured, represented by two connected oscilloscopes, R&S RTH 1044, which was proved by the measurements provided in graphs in FIG. 8 and FIG. 9 respectively. A hydrophone was connected to the system to verify the signal from the transducer.

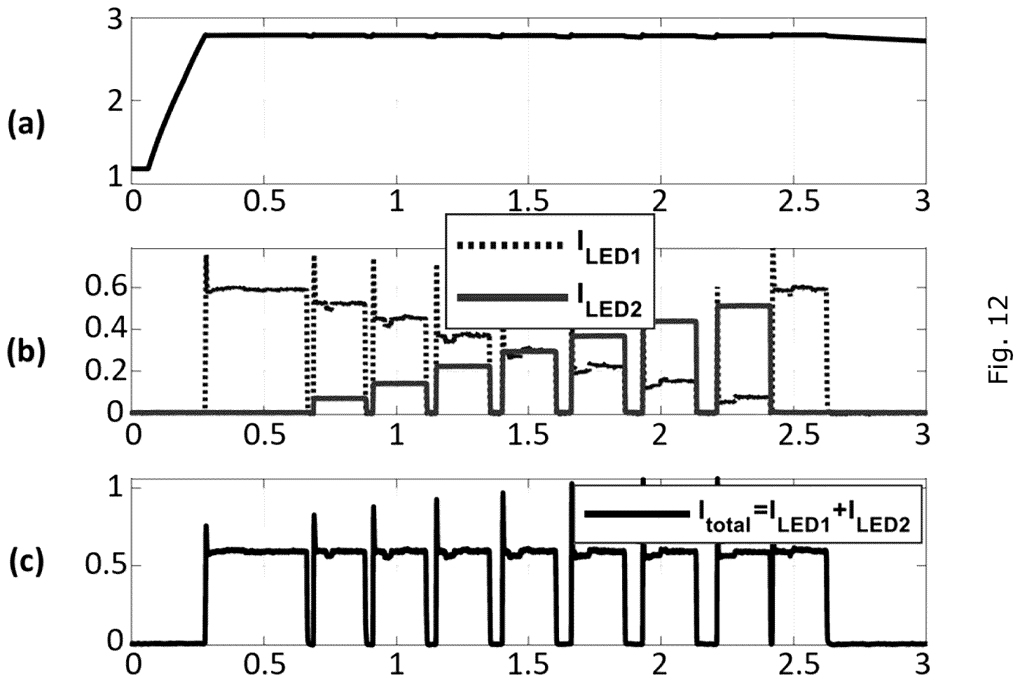

FIG. 12 shows three graphs representing transient measurement results from the experimental set up as shown in FIG. 7.

The upper graph (a) shows Vrec at the micro devices M_D output, with the y-axis representing voltage and the x-axis representing time in milliseconds.

The middle graph (b) shows electric current of I_{LED1}, dotted line and I_{LED2}, solid line, at the micro devices M_D output, with the y-axis representing current in milliamps and the x-axis representing time in milliseconds.

The lower graph (c) shows total load current for I_{LED1} and I_{LED2}, at the micro devices M_D output, with the y-axis representing current in milliamps and the x-axis representing time in milliseconds.

As shown in (b), I_{LED2} increases stepwise from 0 to 514 μA with steps of 74 μA +/−5%, according to the encoded commands over the notch durations, while I_{LED1} takes the rest of the current budget. Thus, VRec, and I_{Total}=I_{LED}+I_{LED2} are regulated to 2.79V, and 600 μA, respectively.

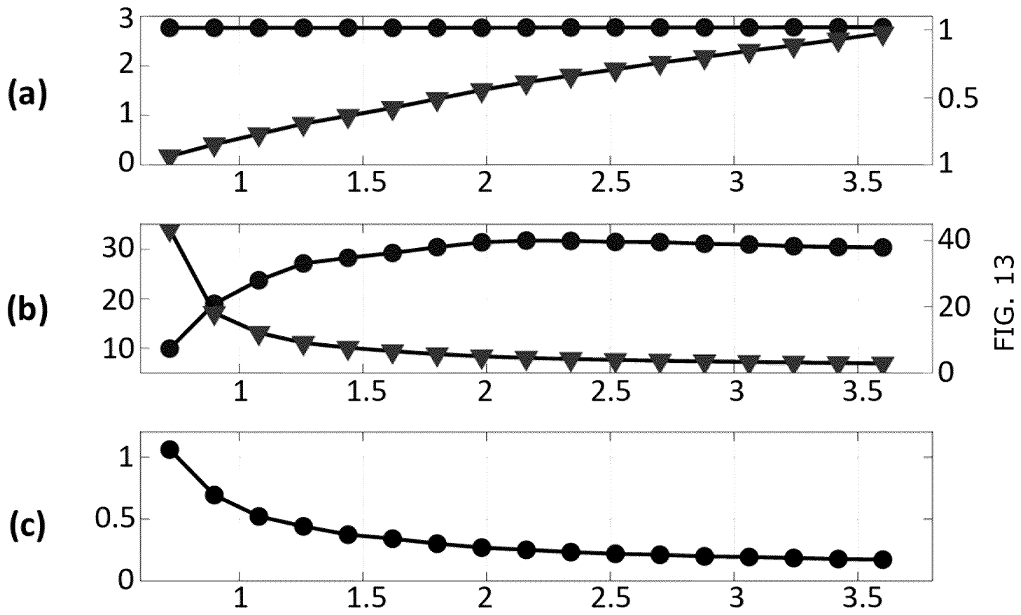

FIG. 13 shows three graphs (a), (b), (c) representing measured acoustic intensity at the piezoelectric receiver (in mW/mm²) P_R, from the experimental set up as shown in FIG. 7. In these measurements, time-average intensity of 2.5 ms ultrasonic power bursts are swept from 0.72 to 3.6 mW/mm² with steps of 0.18 mW/mm². The voltage Vrec and DC current through LED1, and startup time have been measured. The upper graph (a) shows Vrec voltage (in V indicated to the left, shown with circles) and corresponding DC current (in mA indicated to the right, shown with triangles). As the acoustic intensity increases, current through LED1 increases while Vrec stays regulated to 2.79 V±0.5%. Thus the DC resistance decreases. The middle graph (b) shows the efficiency (electrical power at LED1 divided by the acoustic power at the piezo surface) shown with circles in % to the left, and the DC electrical load is shown with triangles (in kΩ) to the right. The lower graph (c) shows that startup time (in ms) of the chip reduces non-linearly by increasing the acoustic power.

To sum up, the invention provides a micro device, such as a brain dust, arranged for implantation into biological tissue. A power management unit receives a wireless signal, e.g. an ultrasonic signal, from an external source and generates an electric power output accordingly. The micro device has a plurality of electrical power consuming components powered by the electric power output, especially comprising one or more controllable light sources, e.g. micro LEDs which can be controlled to generate light in a time-varying manner, such as for optogenetics or for optical release of a drug. To increase power efficiency, an electric regulator circuit regulates electric current applied to the controllable light source to provide a predetermined total electric load of the power management unit. This provides an optimal efficiency of transferring power to the plurality of electrical power consuming components with a minimal requirement of volume of the circuit which provides impedance matching for optimal efficiency.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1-23. (canceled)

24. A micro device arranged for implantation into biological tissue, the micro device comprising:
a power management unit arranged to receive a wireless signal, such as an ultrasonic signal, from an external source and to generate an electric power output accordingly;
a plurality of electrical power consuming components powered by the electric power output, the power consuming components comprising at least one controllable light source arranged to generate light in a controllable time-varying manner; and
an electric regulator circuit to regulate electric current applied to the at least one controllable light source so as to provide a predetermined total electric load of the power management unit for optimal efficiency of transferring power to the plurality of electrical power consuming components.

25. The micro device according to claim 24, wherein the electric regulator circuit is arranged to regulate electric current applied to the at least one controllable light source in a closed-loop manner, so as to regulate a total current applied to all of the electrically power consuming components.

26. The micro device according to claim 24, comprising an impedance matching circuit connected to the power management unit to provide optimal power efficiency, wherein the impedance matching circuit has fixed components.

27. The micro device according to claim 24, wherein the electrical regulator circuit is arranged to disable at least one power consuming component when there is lack of power for energy longevity management at the micro device.

28. The micro device according to claim 24, comprising a wireless receiver separate from the power management unit and being arranged to receive data for controlling at least one of the plurality of electrically power consuming components.

29. The micro device according to claim 24, wherein the at least one controllable light source is arranged for optogenetic stimulation.

30. The micro device according to claim 24, wherein said plurality of electrical power consuming components comprises a second controllable light source, wherein the first and second controllable light sources are arranged to generate light at different wavelengths.

31. The micro device according to claim 30, wherein the first controllable light source comprises a Light Emitting Diode arranged to generate blue light, and wherein the second controllable light source comprises a Light Emitting Diode arranged to generate red light.

32. The micro device according to claim 31, wherein the first and second controllable light sources are arranged for dual color optogenetics.

33. The micro device according to claim 30, wherein the first controllable light source is arranged for one of: optogenetics, photodynamic therapy or for optical triggering of drug delivery, and wherein the second controllable light source is arranged for one of:
optogenetics, photodynamic therapy or for optical triggering of drug delivery.

34. The micro device according to claim 30, wherein the electric regulator circuit is arranged to regulate electric current applied to both of the first and second controllable electric light sources so as to provide the predetermined total electric load of the power management unit for optimal power efficiency.

35. The micro device according to claim 30, wherein the electric regulator circuit comprises a load regulator arranged to drive the first controllable light source, and a current Digital to Analog Converter arranged to drive the second controllable light source.

36. The micro device according to claim 35, wherein the load regulator is arranged to adapt a current to the first controllable light source in order to provide said predetermined total electric load of the power management unit, and/or wherein the load regulator is arranged to drive the first controllable light source, when electric power is available from the power management unit, and/or wherein the load regulator is arranged to drive the second controllable light source based on a received command.

37. The micro device according to claim 24, comprising a third controllable light source arranged for optogenetics, photodynamic therapy or for optical triggering of drug delivery, preferably wherein the first and second controllable light sources are arranged for optogenetics or photodynamic therapy at different wavelengths of light, and wherein the third controllable light source is arranged for optical triggering of drug delivery.

38. The micro device according to claim 24, comprising an electrode arranged for controllable electric stimulation of biological tissue, and wherein the at least one controllable light source is arranged for optogenetics or photodynamic therapy.

39. The micro device according to claim 24, wherein the plurality of electrically power consuming components comprises a sensor arranged to measure neural activity and/or a physical parameter of the biological tissue, such as temperature or pressure.

40. The micro device according to claim 24, wherein the power management unit and the electric regulator circuit are implemented on an integrated circuit die.

41. The micro device according to claim 24, wherein the power management unit comprises a piezoelectric transducer arranged to receive an ultrasonic signal and to generate the electric power output accordingly.

42. The micro device according to claim 24, having a total volume of less than 1 mm³, such as less than 0.5 mm³, such as less than 0.2 mm³.

43. A method for managing power consumption in a micro device arranged for implantation into biological tissue, the method comprising:
providing a wireless signal from an external source to a power management unit of the micro device;
powering electrically power consuming components of the micro device based on electric power from the power management unit; and
regulating an electric current applied to at least one controllable light source using an electric regulator circuit so as to provide a predetermined total electric load of the power management unit for optimal power efficiency.
